(19) United States
(12) Patent Application Publication
(10) Pub. No.: US 2020/0229741 A1
(43) Pub. Date: Jul. 23, 2020

(54) System and Method of Evaluating and Treating Diabetes

(71) Applicants: Adnan Ali Hyder, Lutherville, MD (US); Kashif Latif, Germantown, TN (US)

(72) Inventors: Adnan Ali Hyder, Lutherville, MD (US); Kashif Latif, Germantown, TN (US)

(21) Appl. No.: 16/751,200

(22) Filed: Jan. 23, 2020

Related U.S. Application Data
(60) Provisional application No. 62/795,763, filed on Jan. 23, 2019.

(51) Int. Cl. A61B 5/145; A61B 5/00; G16H 20/10; G16H 50/30

(52) U.S. Cl. CPC A61B 5/14532; A61B 5/7275; G16H 50/30; A61B 5/7282; G16H 20/10; A61B 5/4848

(57) ABSTRACT

A system and method of evaluating and treating diabetes ensures a consistent and enduring path for overcoming diabetes and maintaining a healthy lifestyle. The system required in order to implement the method includes at least one remote server and a corresponding personal (PC) computing device. At least one user account is managed by the at least one remote server and is associated with a corresponding PC device. The overall process begins with a diabetes diagnosis process that determines which type of diabetes is associated with user account. A medical assessment process and a self-management process are interchangeably executed after the diabetes diagnosis process. A risk stratification process then determines how to treat the user account. A clinical prescription process follows, then a behavioral health process, a continuous monitoring process, and a professional follow-up process are interchangeably executed after.

The current application claims priority to U.S. provisional application Ser. No. 62/795,763 filed on Jan. 23, 2019.

### FIELD OF THE INVENTION

The present invention generally relates to evaluating and treating diabetes. More specifically, the present invention systematically treats diabetes and encourages sustainable behaviors that significantly lessen or eliminate diabetes symptoms.

### BACKGROUND OF THE INVENTION

Various medical protocols have been provided over the years to help diagnose and treat diabetes to various degrees of success. However, few wholistic medical protocols for diabetes evaluation and treatment have been provided which include diagnosis, medical history assessment, nutrition and self-management, risk stratification, personalized medicine, behavioral health, continuous monitoring, episode of care follow-up, and feedback into the population health.

An objective of the present invention is to provide evidence-based protocols for wholistic evaluation and treatment of patients with diabetes. The present invention provides a comprehensive assessment and management tool which preferably comprises nine steps. Each step of the present invention is based on years of clinical practice and data from hundreds of patients successfully managed by clinicians. A plurality of specific actions dictates the completion of each step including a quality improvement process integrated across the steps with proactive feedback into population health. The present invention can be integrated into a health care system, a medical facility, or similar systems which provide health care, medical assessment, or medical treatment.

### BRIEF DESCRIPTION OF THE DRAWINGS

FIG. 1 is a block diagram of a system for the present invention.

FIG. 2 is a flowchart illustrating the overall process for a method of the present invention.

FIG. 3 is a flowchart illustrating the interchanging of given steps based on the individual condition and status of a user account.

FIG. 4 is a flowchart illustrating the subprocess of diabetes diagnosis for the present invention.

FIG. 5 is a flowchart illustrating the subprocess of medical assessment for the present invention.

FIG. 6 is a flowchart illustrating the subprocess of self-management process for the present invention with a diabetes information class

FIG. 7 is a flowchart illustrating the subprocess of self-management process for the present invention with a diet-and-fitness plan.

FIG. 8 is a flowchart illustrating the subprocess of risk stratification for the present invention.

FIG. 9 is a flowchart illustrating the subprocess of clinical prescription for the present invention.

FIG. 10 is a flowchart illustrating the subprocess for behavioral health for the present invention.

FIG. 11 is a flowchart illustrating the subprocess of continuous monitoring for the present invention.

FIG. 12 is a flowchart illustrating a continuation of the subprocess of continuous monitoring for the present invention with an insulin pump in order to adjust a glucose-level deviation.

FIG. 13 is a flowchart illustrating the subprocess of follow-up for the present invention.

FIG. 14 is a flowchart illustrating the subprocess of communicative feedback from other users and medical contacts for the present invention.

### DETAIL DESCRIPTIONS OF THE INVENTION

All illustrations of the drawings are for the purpose of describing selected versions of the present invention and are not intended to limit the scope of the present invention.

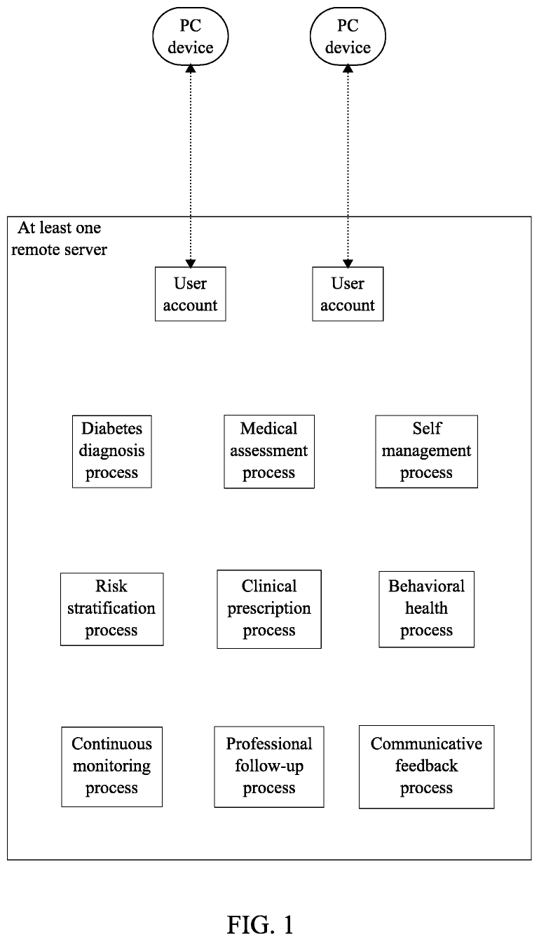

The present invention is a system and method of evaluating and treating diabetes. The innovative method includes an individualized approach to diabetes mellitus. The present invention achieves optimal blood glucose control which minimizes risks of long-term and short-term complications. Thus, the physical system used to implement the method for the present invention includes at least one remote server and a corresponding personal computing (PC) device, seen in FIG. 1. At least one user account is managed by the at least one remote server, wherein the user account is associated with the corresponding PC device (Step A). In the preferred embodiment of the present invention, the at least one remote server is an integral part of a healthcare electronic system or medical facility electronic system. Any data associated with the at least one user account is stored on the at least one remote server. The user account corresponds to an individual suffering from diabetes. Each user account is able to access and adjust associated data with the corresponding PC device. Furthermore, each user account is preferably assessed by a medical professional in order to gain access to the overall process of the present invention. It is preferable, that the user account is able to fulfill certain criteria that statistically show a success rate for the user account. Such criteria may include, but is not limited to, personal information, medical history, referrals from previous medical providers, and insurance and financial information.

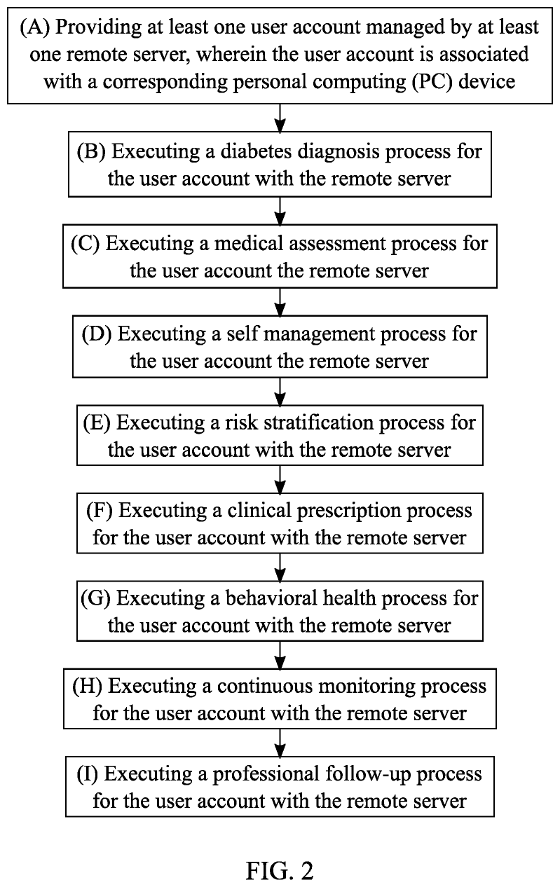

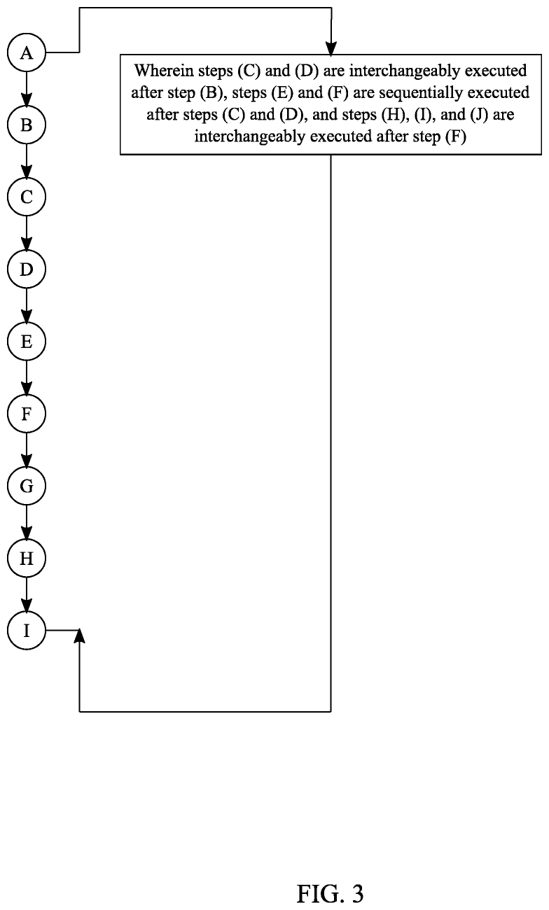

The overall process for the method of the present invention includes the following steps that are implemented with the at least one remote server and the corresponding PC device. As seen in FIG. 2, the overall process begins by executing a diabetes diagnosis process for the user account with the remote server (Step B). The diabetes diagnosis process determines which type of diabetes an individual has, specifically Type I or Type II diabetes, in order to perform the appropriate treatments, as well as monitoring and examinations. A medical assessment process is then executed for the user account with the remote server (Step C) to ensure any past or existing medical and physical conditions do not hinder the progress of the treatment of diabetes. In order to ensure the awareness and increase the knowledge of diabetes, a self-management process is executed for the user account with the remote server (Step D). Furthermore, the nutrition of an individual with diabetes is planned, executed, and monitored with the self-management process. A risk stratification process is then executed for the user account with the remote server (Step E) in order to apply the appropriate standards, treatment requests, and recovery projections with the user account. Moreover, the risk stratification step provides an assessment for the duration of diagnosis and accounts for co-morbidities, family history, and lifestyle factors. Recovery with medication is effectively recommended and applied as a clinical prescription process is executed with the remote server (Step F). The clinical prescription process accounts for cholesterol and blood pressure, specifically, as both may alter the effects of recommended mediation. The maintenance of mental health ensures consistent progress during and after treatment of diabetes with the present invention as a behavioral health process for the user account is executed with the remote server (Step G). A continuous monitoring process is executed for the user account with the remote server in order to monitor physiological factors of an individual (Step H). Data retrieved throughout the continuous monitoring process may be sent, received, and viewed with the corresponding PC device in personal spaces or public medical facilities. In order to determine a full recovery from diabetes and the progress throughout the treatment of diabetes over a given amount of time, a professional follow-up process for the user account is executed with the remote server (Step I). The follow-up process confirms the completion of treatment as well as provide confirmation the completion of checkups and treatment on an as-needed basis. As can be seen in FIG. 3, the present invention is able to successfully treat diabetes with each user account, individually, as Step C and Step D are interchangeably executed after Step B, Step E and Step F are sequentially executed after Step C and Step D, and Step H, Step I, and Step J are interchangeably executed after Step F. Moreover, depending on the severity of the condition of diabetes for the user account and the pre-existing conditions for the user account, the order of execution may vary accordingly.

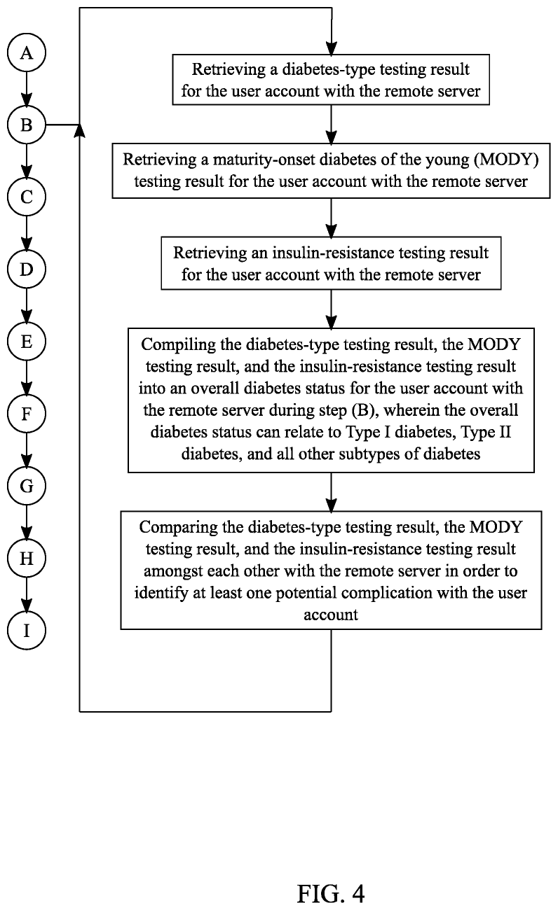

During the diabetes diagnosis process seen in FIG. 4, the type of diabetes associated with the user account is determined as a diabetes-type testing result is retrieved for the user account with the remote server. The diabetes-type testing result provides information regarding which type of diabetes associated with the user account. As Type II diabetes is commonly diagnosed instead of Type I diabetes, a maturity-onset diabetes of the young (MODY) testing result is retrieved for the user account with the remote server. The MODY testing result eliminates any uncertainty regarding the type of diabetes associated with the user account. An insulin-resistance testing result is also retrieved for the user account with the remote server. The insulin-resistance testing result is a standard examination to determine how well the user's body is responding to insulin. The diabetes-type testing result, the MODY testing result, and the insulin-resistance testing result is compiled into an overall diabetes status for the user account with the remote server during Step B, and the overall diabetes status is a comprehensive metric describing the user's health in relation to diabetes. The diabetes-type testing result, the MODY testing result, and the insulin-resistance testing result is compared amongst each other with the remote server in order to identify at least one potential complication with the user account. The potential complication is any future health problem resulting from the user's health in relation to diabetes. In addition, the diabetes diagnosis process can be used for Type I diabetes, Type II diabetes, and all other subtypes of diabetes.

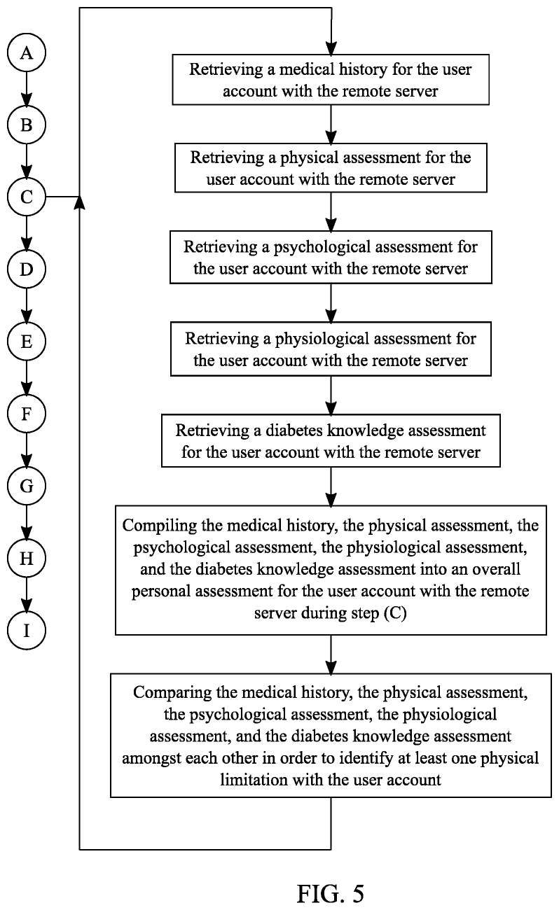

The medical assessment process begins by retrieving a medical history for the user account with the remote server, as seen in FIG. 5. The medical history for the user account may include, but is not limited to, prior surgeries, prior injuries, prior conditions, and family history. A physical assessment, a psychological assessment, and a physiological assessment are retrieved for the user account with the remote server in order to determine any current physical issues, psychological issues, and physiological issues that may hinder the effectiveness of the application of the present invention. More specifically, the physical assessment, the psychological assessment, and the physiological assessment together gather comprehensive data through various examinations such as, Exercise Tolerance (ETT) test, biological assessment, Ankle-Brachial Index (ABI) test, Electrocardiogram (EKG) test, temporary perception assessment, cognitive assessment with tools for genitive assessment, and longitudinal monitoring. A diabetes knowledge assessment is also retrieved for the user account with the remote server in order to determine a baseline of diabetes knowledge for the user account. The medical history, the physical assessment, the psychological assessment, the physiological assessment, and the diabetes knowledge assessment are compiled into an overall personal assessment for the user account with the remote server during Step C, and the overall personal assessment is a comprehensive assessment of the user's ability to manage and/or cure themselves from diabetes. The medical history, the physical assessment, the psychological assessment, the physiological assessment and the diabetes knowledge are preferably integrated with other Electrical Medical Records (EMRs) of a medical facility and the like. The medical history, the physical assessment, the psychological assessment, the physiological assessment, and the diabetes knowledge assessment are compared amongst each other in order to identify at least one physical limitation with the user account. The at least one physical limitation hinders the rate and quality of recovery for the user account. Once the at least one physical limitation is identified, medical professionals are able to address the at least one physical limitation in order to facilitate treatment of diabetes for the user account.

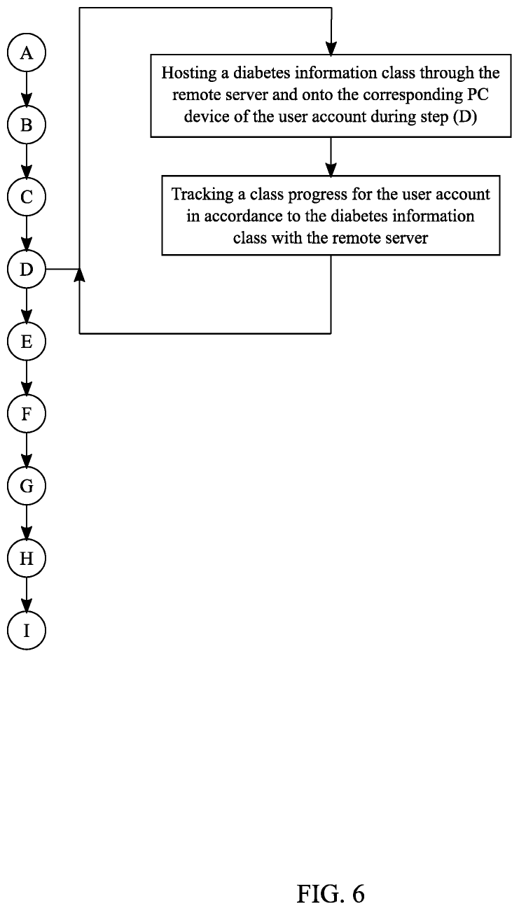

In order to ensure the user account is fully aware of future possibilities with a given diabetes condition and ensure a consistent application of the treatment with the self-management process, a diabetes information class is hosted through the remote server and onto the corresponding PC device of the user account during Step D, as seen in FIG. 6. The diabetes information class provides the user account with essential information regarding diabetes that is specific to the current situation associated with the user account. Certified diabetes educators, registered dietitians, licensed therapists, and so on, may be authorized and present the user account with a set of information that is contoured specifically to the current situation associated with the user account. A class progress is tracked for the user account in accordance to the diabetes information class with the remote server. The class progress provides the success rate of educating the user account with the necessary information in order to aid the treatment of diabetes with the present invention. It is understood that the user account may be presented with the necessary information through the corresponding PC device. The user account may also select available sessions from a schedule of sessions for in-person diabetes information classes.

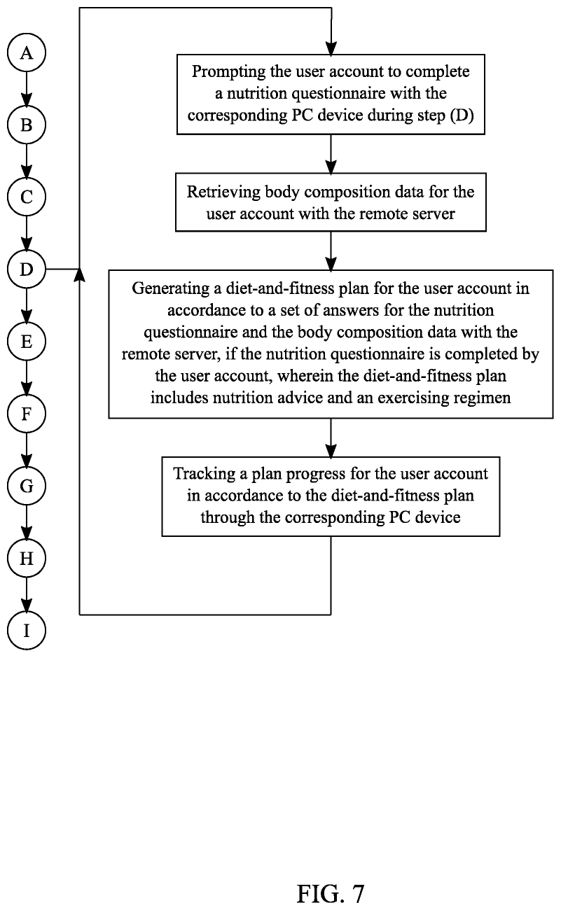

Furthermore, during the self-management process, the user account is prompted to complete a nutrition questionnaire with the corresponding PC device during Step D, as seen in FIG. 7. As nutrition plays a key role in the success of the treatment of diabetes, the nutrition questionnaire determines if the user account is aware of any nutrition limitations while being affected by diabetes and the specific type of diabetes associated with the user account. Body composition data is retrieved for the user account with remote server to determine a baseline for the current physical well-being with the user account. When the nutrition questionnaire is completed by the user account, a diet-and-fitness plan is then generated for the user account in accordance to a set of answers for the nutrition questionnaire and the body composition data with the remote server. The diet-and-fitness plan provides the user account with the necessary information to eliminate any negative effects from foods or ways-of-life. Moreover, the diet-and-fitness plan includes nutrition advice and an exercising regimen. The nutrition plan preferably suggests the foods that are available for the user account based on the current condition of the user account. The exercising regimen suggests a workout routine that may actively lower any high risks associated with diabetes. In order to determine the success of the treatment and eliminate any other possible factors that may hinder the treatment or increase the symptoms diabetes, a plan progress is tracked for the user account in accordance to the diet-and-fitness plan through the corresponding PC device. The plan progress records the activity of the user account with respect to the nutrition advice and the exercising regimen and analyzes the effects of the treatment based on the consistency of the user account.

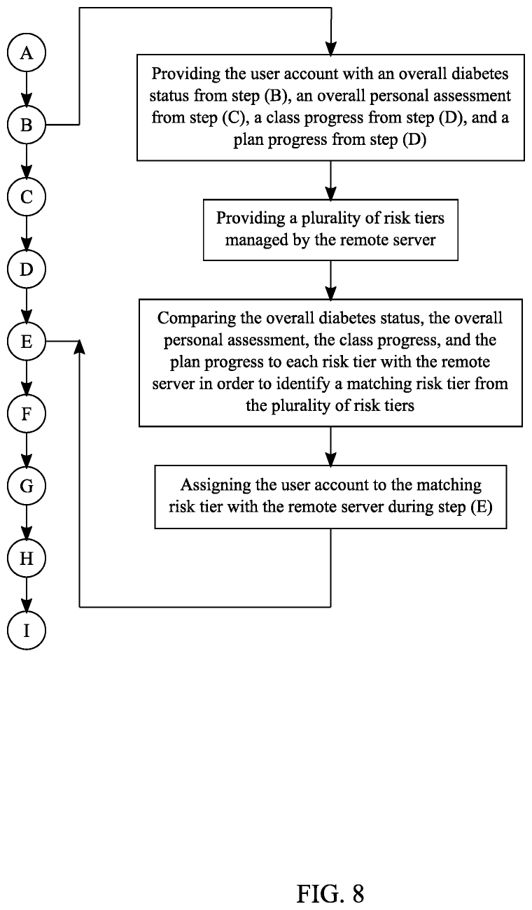

As can be seen in FIG. 8, in order to apply the proper treatment based on the type of condition for the user account, the user account is provided with an overall diabetes status from Step B, an overall personal assessment from Step C, a class progress from Step D, and a plan progress from Step D. The overall diabetes status from Step B defines the type of diabetes associated with the user account. The overall personal assessment from Step C defines the current physical condition for the user account. The class progress from Step D defines the baseline of knowledge and awareness of diabetes with the user account before and during diabetes information class. The plan progress from Step D describes the user's commitment to their diabetes treatment. A plurality of risk tiers is also provided by the remote server, and the plurality of risk tiers is increasing severity levels for the risk of diabetes. The plurality of risk tiers is mainly dependent on, but are limited by, Hemoglobin A1C comparison, risk of Cardiovascular (CV) events, risk of Peripheral Vascular Disease (PVD), risk of Cerebrovascular Accident (CVA), and Framingham Risk Score. The overall diabetes status, the overall personal assessment, the class progress, and the plan progress is compared to each risk tier with the remote server in order to identify a matching risk tier from the plurality of risk tiers. Each matching risk tier is associated with specific diabetic conditions and requirements that facilitate the recovery for the user account. The user account is assigned to the matching risk tier with the remote server during Step E in order to deliver a variety of requests for user account with the corresponding PC device.

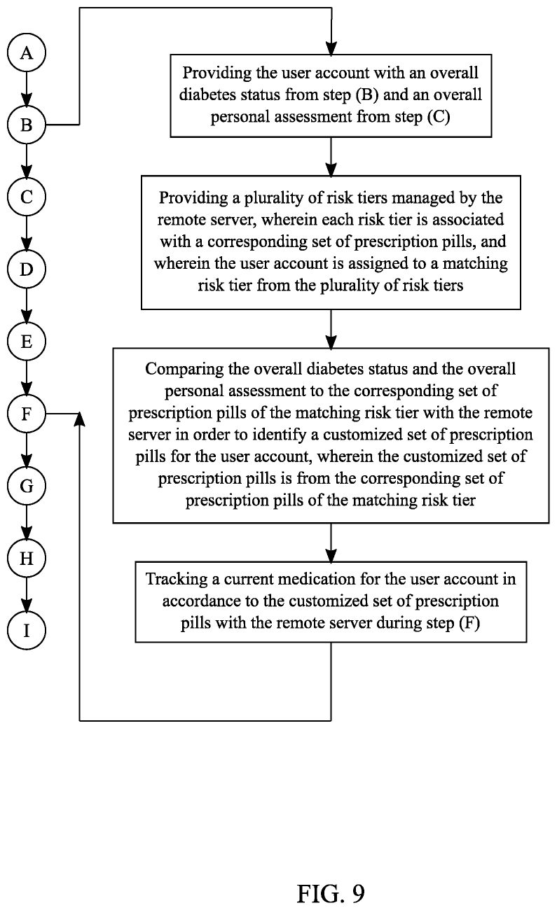

Each user account may proceed with the appropriate treatment with medication after the risk stratification process. As seen in FIG. 9, in order to be notified of the available medication based on the specific condition of the user account, the user account is provided with an overall diabetes status from Step B and an overall personal assessment from Step C. A plurality of risk tiers managed by the remote server is also provided, wherein each risk tier is associated with a corresponding set of prescription pills. The corresponding set of prescription pills reduces the symptoms and preferably eliminate the symptoms associated with the user account based on the type of diabetes of the user account. The corresponding set of prescription pills preferably lowers Hemoglobin A1C while addressing cholesterol and blood pressure concerns. Moreover, the user account is assigned to a matching risk tier from the plurality of risk tiers so that the user account does not receive a set of prescription pills of other risk tiers that may prove harmful or fatal for the current condition of the user account. The overall diabetes status and the overall personal assessment is compared to the corresponding set of prescription pills of the matching risk tier with the remote server in order to identify a customized set of prescription pills for the user account. The customized set of prescription pills is selected from the corresponding set of prescription pills of the matching risk tier in order to cater a selection of prescription pills around the user's specific physiology. A current medication is tracked for the user account in accordance to the customized set of prescription pills with the remote server during Step F. The current medication serves as a record of the medications ingested by the user account in an attempt to relieve and eliminate diabetic symptoms in the event of a relapse.

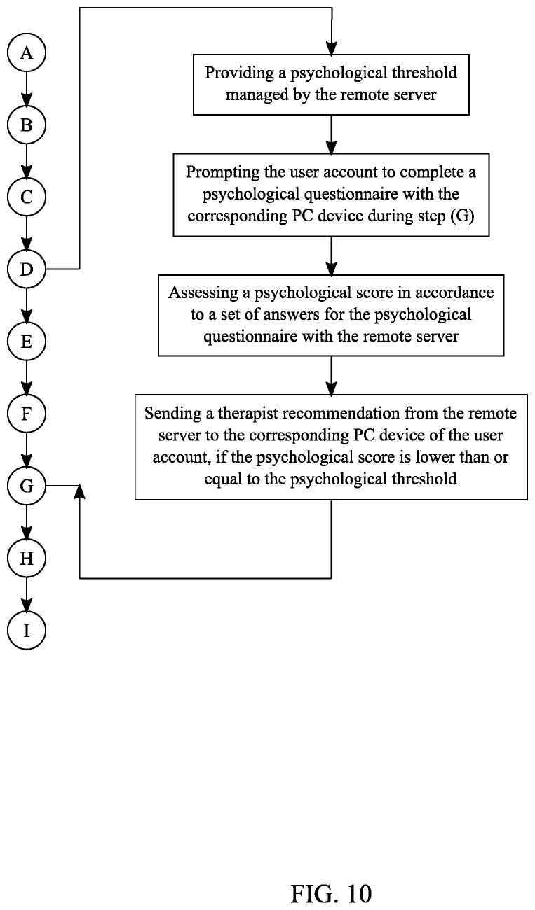

The overall well-being of the user account is monitored in order to facilitate the treatment of diabetes with the present invention with the behavioral health process, shown in FIG. 10. During the behavioral health process, a psychological threshold managed by the remote server is provided. The psychological threshold is the lowest acceptable limit for the state of the mental health of the user account. The user account is prompted to complete a psychological questionnaire with the corresponding PC device during Step G. The psychological questionnaire determines if the mental health of the user account throughout the overall process of the present invention is good, safe, or in danger. A psychological score is assessed in accordance to a set of answer for the psychological questionnaire with the remote server. The psychological score defines whether the mental health of the user account is good, safe, or in danger. A therapist recommendation is sent from the remote server to the corresponding PC device of the user account, if the psychological score is lower than or equal to the psychological threshold. The therapist recommendation includes contact information associated with a therapist that is best capable to help navigate the mental health that is in danger for the user account.

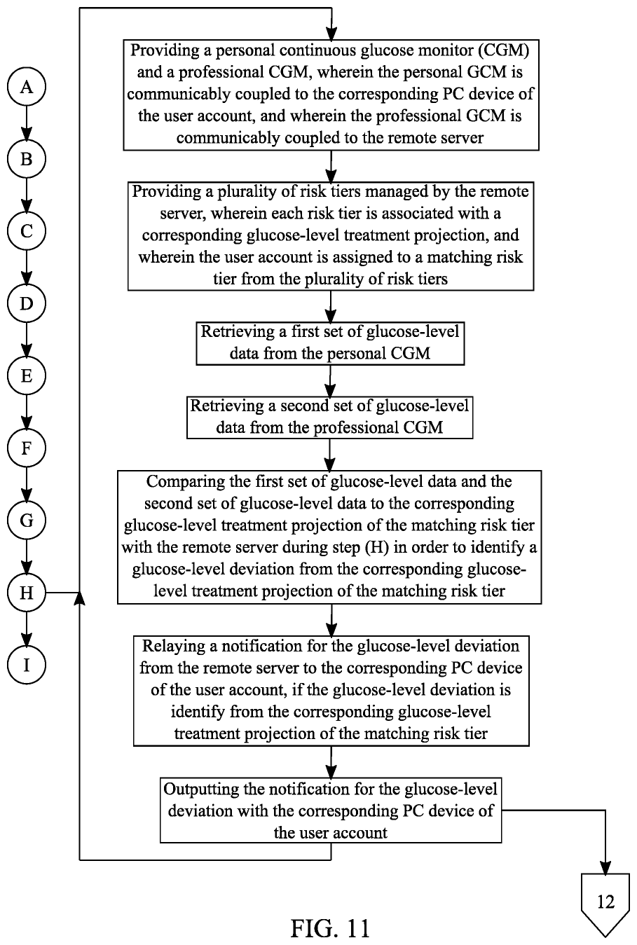

After the risk stratification process, exact and precise test results are required to effectively monitor the progress of the user account with continuous monitoring process, which is shown in FIG. 11. A personal continuous glucose monitor (CGM) and a professional CGM is provided, wherein the personal CGM is communicably coupled to the corresponding PC device of the user account, and the professional CGM is communicably coupled to the remote server. The personal CGM and the professional CGM monitors and measures glucose levels in real-time in a personal setting or a medical professional environment. A plurality of risk tiers managed by the remote server is provided, wherein each risk tier is associated with a corresponding glucose-level treatment projection. The glucose-level treatment projection provides a range of glucose levels that is defined as safe for the user account. Moreover, the user account is assigned to a matching risk tier from the plurality of risk tiers. A first set of glucose-level data is retrieved from the personal CGM, and a second set of glucose-level data is retrieved from the professional CGM. The first set of glucose-level data and the second set of glucose-level data is compared to the corresponding glucose-level treatment projection of the matching risk tier with the remote server during Step H in order to identify a glucose-level deviation from the corresponding glucose-level treatment projection of the matching risk tier. The glucose-level deviation identifies increased or significantly low glucose levels and the severity of increased and significantly low glucose levels with respect to the glucose-level treatment projection. A notification for glucose-level deviation is relayed from the remote server to the corresponding PC device of the user account, if the glucose-level deviation is identified from the corresponding glucose-level treatment projection of the matching risk tier. The notification for the glucose-level deviation alerts the user account of the increased and significantly low glucose levels in an attempt to facilitate immediate action to adjust their glucose levels. The notification for the glucose-level deviation is outputted with the corresponding PC device of the user account. The notification may be a text and or display associated with a sound alert.

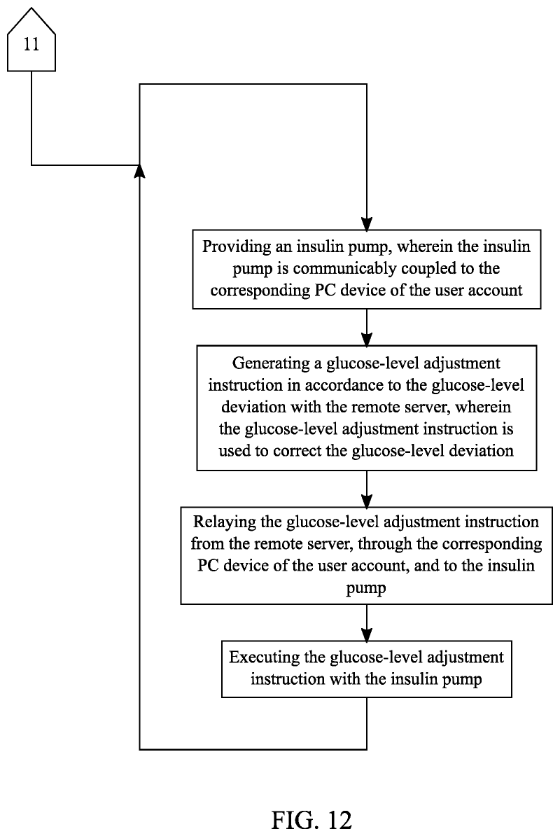

In the event the first set of glucose-level data or the second set of glucose-level data are not within the glucose-level deviation for the corresponding glucose-level treatment projection of the matching risk tier, an insulin pump is provided, as seen in FIG. 12. The insulin pump is communicably coupled to the corresponding PC device of the user account so that the insulin pump may remotely operate in accordance with the remote server. The insulin pump regulates the glucose levels of the user account. Furthermore, the insulin pump optimizes the success rate of the user account to be properly treated. A glucose-level adjustment instruction is generated in accordance to the glucose-level deviation with the remote server, wherein the glucose-level adjustment instruction is used to correct the glucose-level deviation. The glucose-level adjustment instruction directly controls the insulin pump in order to operate the insulin pump based on the conditions specific for the user account. The glucose-level adjustment instruction is relayed from the remote server, through the corresponding PC device of the user account, and to the insulin pump. The glucose-level adjustment instruction is the executed with the insulin pump to correct the glucose-level deviation.

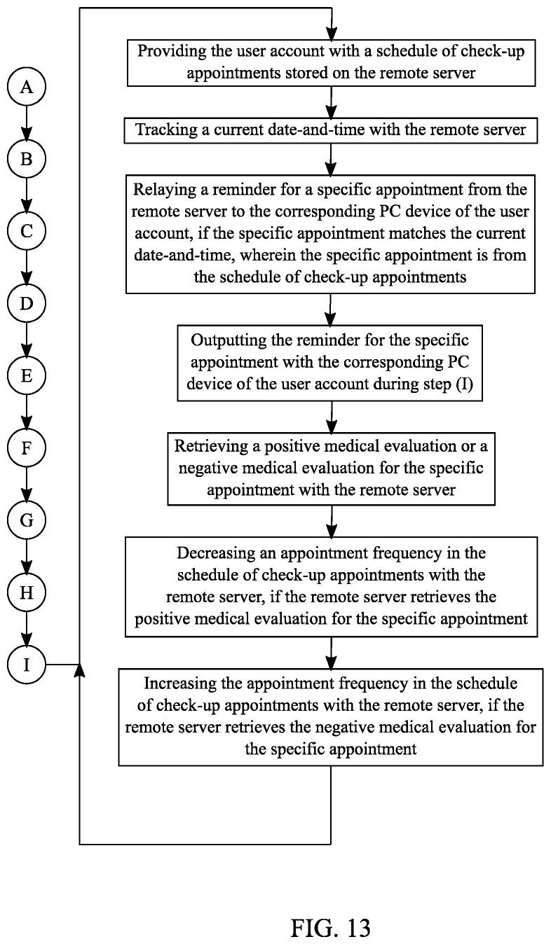

In order to execute the follow-up process, the user account is provided with a schedule of check-up appointments stored on the remote server, as seen in FIG. 13. Each check-up appointments are required check-ins with a medical professional by the user account in order to determine the consistency of the user account with the treatment and the progress of the user account. A current date-and-time is tracked with the remote server in order to alert the user account with the corresponding PC device in real-time of each check-up appointment. In the preferred embodiment of the present invention, cellular communication is enabled with the corresponding PC device in order for the user account to communicate with a medical professional in the event that an in-person appointment is not possible due to physical limitation associated with the user account. A reminder for a specific appointment is relayed from the remote server to the corresponding PC device of the user account, if the specific appointment matches the current date-and-time, wherein the specific appointment is from the schedule of check-up appointments. The reminder for the specific appointment is then outputted with the corresponding PC device of the user account during Step I. The reminder may be a text or display with a sound alert through the corresponding PC device. During each check-up appointment, a variety of exams may be conducted in order to retrieve decreasing Hemoglobin A1C, increased knowledge of Hemoglobin A1C, increased stamina as a result of consistency with the exercise regimen, weight loss, decreased body measurements such as smaller waistline, stabilized blood sugar levels, decreased blood pressure levels, and so on. Annual assessments for vascular status and both EKG and ABI results may also be performed. For user accounts associated with abnormal coronary anatomy, an annual functional assessment may be required with an echocardiogram. For user accounts associated with peripheral vascular disease (PVD), an annual exercise assessment may be required.

Following each check-up appointment of the schedule of check-up appointments, a positive medical evaluation or a negative medical evaluation is then retrieved for the specific appointment with the remote server, also seen in FIG. 12. The positive medical evaluation indicates consistent effort of the user account to follow the overall process of the present invention and/or indicates a positive progression of the overall wellbeing of the user account. The negative medical evaluation indicates inconsistent effort of the user account and/or indicates a negative progression of the overall wellbeing of the user account. An appointment frequency is decreased in the schedule of check-up appointments with the remote server, if the remote server retrieves the positive medical evaluation for the specific appointment. The appointment frequency is the number of check-ins of the user account with a medical professional, and the like, over a given range of time. Conversely, the appointment frequency is increased in the schedule of check-up appointments with the remote server, if the remote server retrieves the negative medical evaluation for the specific appointment.

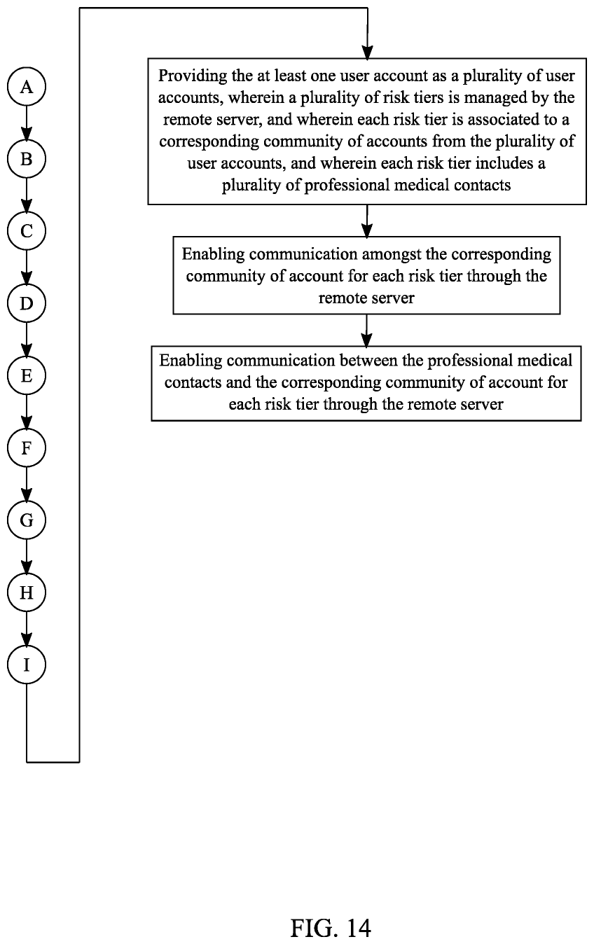

For the communicative feedback process after Step I, the user account is able to consistently maintain a given level of progress throughout each step as the user account is able to easily access other user accounts with comparable conditions and medical professionals, as seen in FIG. 14. Moreover, the at least one user account is provided as a plurality of user accounts, wherein a plurality of risk tiers is managed by the remote server. The plurality of user accounts includes a variety of individuals experiencing diabetes to varying degrees and facing specific limitations. Each risk tier is associated to a corresponding community of accounts from the plurality of user accounts, and each risk tier includes a plurality of professional medical contacts. The plurality of professional medical contacts includes, but is not limited to, a case manager, a dietician, a physical therapist, a physician, and so on. Moreover, the case manager provides services to families associated with a user account with lifestyle intervention and guidance throughout the overall process of the present invention. Communication amongst the corresponding community of the user account is enabled for each risk tier through the remote server. Similarly, communication between the professional medical contacts and the corresponding community of the user accounts is enabled for each risk tier through the remote server.

Although the invention has been explained in relation to its preferred embodiment, it is to be understood that many other possible modifications and variations can be made without departing from the spirit and scope of the invention as hereinafter claimed.

What is claimed is:

1. A method of evaluating and treating diabetes, the method comprises the steps of:
(A) providing at least one user account managed by at least one remote server, wherein the user account is associated with a corresponding personal computing (PC) device;
(B) executing a diabetes diagnosis process for the user account with the remote server;
(C) executing a medical assessment process for the user account with the remote server;
(D) executing a self-management process for the user account with the remote server;
(E) executing a risk stratification process for the user account with the remote server;
(F) executing a clinical prescription process for the user account with the remote server;
(G) executing a behavioral health process for the user account with the remote server;
(H) executing a continuous monitoring process for the user account with the remote server; and,
(I) executing a professional follow-up process for the user account with the remote server.

2. The method of evaluating and treating diabetes, the method as claimed in claim 1, wherein steps (C) and (D) are interchangeably executed after step (B), steps (E) and (F) are sequentially executed after steps (C) and (D), and steps (H), (I), and (J) are interchangeably executed after step (F).

3. The method of evaluating and treating diabetes, the method as claimed in claim 1 comprises the steps of:
retrieving a diabetes-type testing result for the user account with the remote server;
retrieving a maturity-onset diabetes of the young (MODY) testing result for the user account with the remote server;
retrieving an insulin-resistance testing result for the user account with the remote server; and,
compiling the diabetes-type testing result, the MODY testing result, and the insulin-resistance testing result into an overall diabetes status for the user account with the remote server during step (B).

4. The method of evaluating and treating diabetes, the method as claimed in claim 3 comprises the steps of:
comparing the diabetes-type testing result, the MODY testing result, and the insulin-resistance testing result amongst each other with the remote server in order to identify at least one potential complication with the user account.

5. The method of evaluating and treating diabetes, the method as claimed in claim 1 comprises the steps of:
retrieving a medical history for the user account with the remote server;
retrieving a physical assessment for the user account with the remote server;
retrieving a psychological assessment for the user account with the remote server;
retrieving a physiological assessment for the user account with the remote server;
retrieving a diabetes knowledge assessment for the user account with the remote server; and,
compiling the medical history, the physical assessment, the psychological assessment, the physiological assessment, and the diabetes knowledge assessment into an overall personal assessment for the user account with the remote server during step (C).

6. The method of evaluating and treating diabetes, the method as claimed in claim 5 comprises the steps of:
comparing the medical history, the physical assessment, the psychological assessment, the physiological assessment, and the diabetes knowledge assessment amongst each other in order to identify at least one physical limitation with the user account.

7. The method of evaluating and treating diabetes, the method as claimed in claim 1 comprises the steps of:
hosting a diabetes information class through the remote server and onto the corresponding PC device of the user account during step (D); and,
tracking a class progress for the user account in accordance to the diabetes information class with the remote server.

8. The method of evaluating and treating diabetes, the method as claimed in claim 1 comprises the steps of:
prompting the user account to complete a nutrition questionnaire with the corresponding PC device during step (D);
retrieving body composition data for the user account with the remote server;
generating a diet-and-fitness plan for the user account in accordance to a set of answers for the nutrition questionnaire and the body composition data with the remote server, if the nutrition questionnaire is completed by the user account, wherein the diet-and-fitness plan includes nutrition advice and an exercising regimen; and,
tracking a plan progress for the user account in accordance to the diet-and-fitness plan through the corresponding PC device.

9. The method of evaluating and treating diabetes, the method as claimed in claim 1 comprises the steps of:
providing the user account with an overall diabetes status from step (B), an overall personal assessment from step (C), a class progress from step (D), and a plan progress from step (D);
providing a plurality of risk tiers managed by the remote server;
comparing the overall diabetes status, the overall personal assessment, the class progress, and the plan progress to each risk tier with the remote server in order to identify a matching risk tier from the plurality of risk tiers; and,
assigning the user account to the matching risk tier with the remote server during step (E).

10. The method of evaluating and treating diabetes, the method as claimed in claim 1 comprises the steps of:
providing the user account with an overall diabetes status from step (B) and an overall personal assessment from step (C);
providing a plurality of risk tiers managed by the remote server, wherein each risk tier is associated with a corresponding set of prescription pills, and wherein the user account is assigned to a matching risk tier from the plurality of risk tiers;
comparing the overall diabetes status and the overall personal assessment to the corresponding set of prescription pills of the matching risk tier with the remote server in order to identify a customized set of prescription pills for the user account, wherein the customized set of prescription pills is from the corresponding set of prescription pills of the matching risk tier; and,
tracking a current medication for the user account in accordance to the customized set of prescription pills with the remote server during step (F).

11. The method of evaluating and treating diabetes, the method as claimed in claim 1 comprises the steps of:
providing a psychological threshold managed by the remote server;
prompting the user account to complete a psychological questionnaire with the corresponding PC device during step (G);
assessing a psychological score in accordance to a set of answers for the psychological questionnaire with the remote server; and,
sending a therapist recommendation from the remote server to the corresponding PC device of the user account, if the psychological score is lower than or equal to the psychological threshold.

12. The method of evaluating and treating diabetes, the method as claimed in claim 1 comprises the steps of:
providing a personal continuous glucose monitor (CGM) and a professional CGM, wherein the personal CGM is communicably coupled to the corresponding PC device of the user account, and wherein the professional CGM is communicably coupled to the remote server;
providing a plurality of risk tiers managed by the remote server, wherein each risk tier is associated with a corresponding glucose-level treatment projection, and wherein the user account is assigned to a matching risk tier from the plurality of risk tiers;
retrieving a first set of glucose-level data from the personal CGM;
retrieving a second set of glucose-level data from the professional CGM;
comparing the first set of glucose-level data and the second set of glucose-level data to the corresponding glucose-level treatment projection of the matching risk tier with the remote server during step (H) in order to identify a glucose-level deviation from the corresponding glucose-level treatment projection of the matching risk tier;
relaying a notification for the glucose-level deviation from the remote server to the corresponding PC device of the user account, if the glucose-level deviation is identified from the corresponding glucose-level treatment projection of the matching risk tier; and,
outputting the notification for the glucose-level deviation with the corresponding PC device of the user account.

13. The method of evaluating and treating diabetes, the method as claimed in claim 12 comprises the steps of:
providing an insulin pump, wherein the insulin pump is communicably coupled to the corresponding PC device of the user account;
generating a glucose-level adjustment instruction in accordance to the glucose-level deviation with the remote server, wherein the glucose-level adjustment instruction is used to correct the glucose-level deviation;
relaying the glucose-level adjustment instruction from the remote server, through the corresponding PC device of the user account, and to the insulin pump; and,
executing the glucose-level adjustment instruction with the insulin pump.

14. The method of evaluating and treating diabetes, the method as claimed in claim 1 comprises the steps of:
providing the user account with a schedule of check-up appointments stored on the remote server;
tracking a current date-and-time with the remote server;
relaying a reminder for a specific appointment from the remote server to the corresponding PC device of the user account, if the specific appointment matches the current date-and-time, wherein the specific appointment is from the schedule of check-up appointments; and,
outputting the reminder for the specific appointment with the corresponding PC device of the user account during step (I).

15. The method of evaluating and treating diabetes, the method as claimed in claim 14 comprises the steps of:
retrieving a positive medical evaluation or a negative medical evaluation for the specific appointment with the remote server;
decreasing an appointment frequency in the schedule of check-up appointments with the remote server, if the remote server retrieves the positive medical evaluation for the specific appointment; and,
increasing the appointment frequency in the schedule of check-up appointments with the remote server, if the remote server retrieves the negative medical evaluation for the specific appointment.

16. The method of evaluating and treating diabetes, the method as claimed in claim 1 comprises the steps of:
providing the at least one user account as a plurality of user accounts, wherein a plurality of risk tiers is managed by the remote server, and wherein each risk tier is associated to a corresponding community of accounts from the plurality of user accounts, and wherein each risk tier includes a plurality of professional medical contacts;
enabling communication amongst the corresponding community of account for each risk tier through the remote server; and,
enabling communication between the professional medical contacts and the corresponding community of account for each risk tier through the remote server.
